SEQUENCE LISTING (4 sequences)

SEQ ID NO: 1 - DNA, 26 bp, Human
gatgacagatactgtgaaagcatcat

SEQ ID NO: 2 - DNA, 22 bp, Human
agtggacaggtaagccattttc

SEQ ID NO: 3 - DNA, 23 bp, Human
catcacgaagtggtgaagttcat

SEQ ID NO: 4 - DNA, 24 bp, Human
cttgtcttgctctatctttctttg

(19) United States
(12) Patent Application Publication
(10) Pub. No.: US 2015/0038559 A1
(43) Pub. Date: Feb. 5, 2015

(54) Pharmaceutical Composition and Method for the Treatment of Neurodegenerative Diseases, in Particular Amyotrophic Lateral Sclerosis

(71) Applicant: OOO "NTpharma", Moscow (RU)

(72) Inventors: Rustam R. ATAULLAKHANOV, Moscow (RU); Maksim M. SHMAROV, Moscow (RU)

(21) Appl. No.: 14/460,110

(22) Filed: Aug. 14, 2014

Related U.S. Application Data
(63) Continuation of application No. PCT/RU2012/000364, filed on May 11, 2012.

(30) Foreign Application Priority Data
Feb. 16, 2012 (RU) 2012105305

(51) Int. Cl. A61K 48/00; C12N 7/00; C12N 15/86; A61K 38/46; A61K 38/18

(52) U.S. Cl. CPC A61K 48/005; A61K 38/465; A61K 38/1866; C12N 15/86; A61K 48/0075; A61K 48/0083; C12N 7/00; C12N 2999/005; C12N 2710/10071; C12N 2710/10041
USPC 514/44 R

(57) ABSTRACT

A pharmaceutical composition and method for therapy of neurodegenerative diseases including amyotrophic lateral sclerosis, the composition comprising an adenoviral vector, expressing a human angiogenesis factor gene; an effective amount of the adenoviral vector in the form of a non-replicating nanoparticle based on a genome of human adenovirus type 5 with an insertion of a human angiogenin gene which produces the human angiogenesis in a human body; a non-replicating nanoparticle based on the genome of human adenovirus type 5 with an insertion of a human vascular endothelium growth factor gene; and a formulating buffer; wherein the human angiogenin factor gene and the human vascular endothelium growth factor gene are cloned in two expression cassettes within one non-replicating nanoparticle based on the genome of human adenovirus type 5.

### RELATED APPLICATIONS

This Application is a Continuation application of International Application PCT/RU2012/000364, filed on May 11, 2012, which in turn claims priority to Russian Patent Applications No. RU 2012105305, filed Feb. 16, 2012, both of which are incorporated herein by reference in their entirety.

### FIELD OF THE INVENTION

The invention belongs to the sphere of medicine, in particular, to neurology and what concerns the pharmaceutical composition—for therapy of neurodegenerative diseases, in particular, amyotrophic lateral sclerosis.

### BACKGROUND OF THE INVENTION

Amyotrophic lateral sclerosis (ALS, in literature sources the disease is identified as well as Charcot diseases, Lou Gehrig disease, motor neuron disease) is a chronic progressing, clinically and genetically heterogeneous, fatal disease that is characterized by the progressing loss of brain and spinal brain motor neurons. Changes affect cells of anterior horns of the spinal brain (cervical, thoracic and lumbar segments), motor nuclei of the cerebral trunk, pyramidal neurons of the motor zone of the brain cortex. As a result of motor neurons loss the corresponding muscular fibers become denervated and atrophy. The disease is characterized by clinical heterogeneity determined by predominant degeneration of certain subpopulations of motor neurons, due to which it's accepted to identify the bulbar, cervical and thoracic and lumbar-sacral as well as the so-called “high” form of disease. Irrespective of the primary lesion area, the disease with time acquires symmetrical generalized nature.

As a result of steady progressing of ALS the death is caused by breathing paralysis or other complications in 3-5 years on the average after the symptoms manifestation moment.

Currently the etiology of the sporadic form of ALS remained undefined.

At present, no effective ethiopathogenetic methods of ALS therapy are developed. The only preparation registered and available since 1995 for ALS therapy is known, it's based on 6-(trifluoromethoxy)benzothiazol-2-amine and produced under the trademark Rilutek by Sanofi-Aventis (U.S. Pat. No. 5,527,814, Louvel E., 18.06.1996, Use of 2-amino-6-(trifluoromethoxy)benzothiazole for obtaining a medicament for the treatment of amyotrophic lateral sclerosis.). The preparation exerts multifaceted effect on the mechanism of the glutamate neurotransmission. But the issue about its clinical benefit due to high cost and low effectiveness remains. This preparation prolongs the life expectancy of patients by 2-3 months at the average. The drawback of the therapy by Rilutek is the necessity of its double peroral administration (by 50 mg each 12 hours) for the life term at the same time the preparation doesn't improve the muscular function and doesn't decelerate the disease symptom development (Miller R. G. et. al., Riluzole for amyotrophic lateral sclerosis (ALS), motor neuron disease (MND), Cochrane Database Syst Rev (1): CD001447, doi:10.1002/14651858.CD001447.pub2. PMID 17253460, 2007).

Detection of the neutrophic function in well-known angiogenesis factors—the angiogenesis factor (Greenway M. J. et al., ANG mutations segregate with familial and ‘sporadic’ amyotrophic lateral sclerosis, Nat. Genet., 2006; No. 38, p. 411-413), as well as the vascular endothelium growth factor (Lambrechts D. et al., VEGF is a modifier of amyotrophic lateral sclerosis in mice and humans and protects motor neurons against ischemic death, Nat. Genet., 2003, No. 34, p. 383-394) allows considering neurotrophic factors as the most perspective compounds for ALS therapy (Zavalishin I. A., Zakharova M. N. Neurodegenerative diseases and ageing, Moscow, 2001, p. 354-399; Amyotrophic lateral sclerosis, ed. Zavalishin I. A., M, Evrasia, 2007, p. 354-423.).

The importance of the angiogenesis factor in ALS pathogenesis is confirmed in experiments on mice of B6SJL-Tg(SOD1*G93A)dl1Gur/J line with G93A mutation in SOD1 fene (a verified transgenic ALS model) (Gurney M. E. et al., Motor neuron degeneration in mice that express a human Cu,Zn superoxide dismutase mutation, Science, 1994, No. 264, p. 1772). Life expectancy prolongation was demonstrated following administration of the recombinant angiogenesis factor to them (US Patent Application, No. 2008/0045456.).

Involvement of the vascular endothelial growth factor in ALS pathogenesis is confirmed experimentally as well. Deletion of the promoter element of the vascular endothelium growth factor gene determining the reaction for hypoxia, transgenic mice demonstrate development of the lower motor neurons lesion syndrome resembling ALS (Oosthuyse et al., Deletion of the hypoxia—response element in the vascular endothelial growth factor promoter causes motor neuron degeneration, Nat. Genet.-2001, No. 2, p. 131-138).

Pharmaceutical compositions on the basis of the human angiogenesis factor, including the one of the recombinant origin (obtained in E. coli), and methods of their use for therapy of neurodegenerative diseases, in particular ALS, are known that allow reducing manifestations of neurodegenerative processes in experimental mice and prolonging their life expectancy (US Patent Application 2008/0045456.).

The disadvantages of these [symbol] pharmaceutical compositions include the necessity of daily injection of the recombinant angiogenesis factor (as it's a finished protein). The therapy cost is high as well.

Viral vectors are one of the most perspective and effective methods of the genetic material delivery to organs and target cells. Due to lack of ability to integrate with the host genome the adenoviral system provides provisional transgene expression requiring repeated injection of viral constructions with the therapeutic purpose. Important factors include: safety, low toxicity of adenoviral vectors and a high transgene expression in target cells. These vectors expressing various therapeutic neurotrophic factors were successfully applied on various animal models (Miagkov et al., Gene transfer of baculoviral p35 by adenoviral vector protects human cerebral neurons from apoptosis, DNA and cell biology, No. 23 (8), 2004, p. 496-501).

There are known pharmaceutical compositions on the basis of neurotrophic factors one of which is the vascular endothelial growth factor and methods of their application improving the neuron survival. Production of the human recombinant vascular endothelium growth factor is possible with the help of viral vectors, including adenoviral ones. Experiments were conducted on mice (US Patent Application 2011/0212055.).

There is a known ALS therapeutic method with the help of the recombinant angiogenesis factor that was subject to preclinical mice trials, with G93A mutation in the SOD1 gene, as a result of which angiogenin was injected in the spinal cord and prolongation of life span was observed, as well as reduction of carcinogenic effect systemic injection. Mice therapy for 50 days prolonged the life duration from 127 to 138 days (US Patent Application 2008/0045456.).

There are known pharmaceutical compositions and methods of application of the angiogenesis factor as well, including those with use of viral vectors that reduce, improve the course or decelerate one or more of the inherent ALS symptoms:
motor neuron degeneration;
muscular weakness;
muscular atrophy;
involuntary muscular contractions;
frontal and temporal mental debility;
life duration reduction.

The experiments were performed on mice.

(US Patent Application 2011/0078804.). A therapeutic use of the recombinant angiogenesis factor is the closest prevention and/or therapeutic method of neurodegenerative diseases, in particular, ALS of the same intended use as the claimed invention by the combination of characteristics. In accordance with the known method of the angiogenesis factor administration in the expression adenoviral construction is performed to mice by various methods in the range of dosages 0.0001-30 mg/kg of body weight, which allows improving motor functions of muscles and prolonging life.

The pharmaceutical composition and the method of use on the basis of the adenoviral vector expressing the human angiogenesis factor gene was chosen by the authors as the prototype as the closest solution to the claimed invention by the combination of characteristics and the intended use serving for therapy of neurodegenerative diseases, ALS in particular.

Reasons preventing achievement of the therapeutic effect with use of these compositions by the prototype include:
a low therapeutic effect;
no therapeutic dosages for human use;
no therapeutic dosage safety data for human use;

Though studies of the above-stated compositions containing either recombinant angiogenesis factor or the recombinant vascular endothelial growth factor showed promising results for ALS therapy, their significant disadvantages include only minor reduction of symptoms and prolongation of the life duration of experimental animals as well as no clinical trials.

In accordance with the data provided above the investigation of the new pharmacological compositions on the basis of neurotrophic factors as well as development of methods of their use for therapy and/or prevention of human ALS is an urgent and modern task as the medicine is in acute need of safe and economically advantageous preparations not only considerably prolonging the patient life but improving its quality due to reduction or removal of symptoms and signs.

### SUMMARY OF THE INVENTION

The task of this invention is development of the biologically safe, therapeutically effective, convenient for use and economically advantageous pharmaceutical composition with neurotrophic effect, and the development of a certain method of use of the pharmaceutical composition for therapy of neurodegenerative diseases, amyotrophic lateral sclerosis in particular.

The task is carried out through production of a pharmaceutical composition for therapy of neurodegenerative diseases, amyotrophic lateral sclerosis in particular, containing an adenoviral vector expressing the human angiogenesis factor gene, at the same time it contains in the effective amount an adenoviral vector in the form of the human adenovirus type 5 genome based non-replicating nanoparticle with the human angiogenesis factor gene insert producing in the human body the angiogenesis factor, and the human adenovirus type 5 genome based non-replicating nanoparticle with the human vascular endothelium growth factor gene insert producing in the human body the vascular endothelial growth factor, at the same time the human angiogenesis factor gene and the human vascular endothelium growth factor gene are cloned in two expressing cassettes of a single human adenovirus type 5 genome based non-replicating nanoparticle and the composition additionally contains a formulating buffer. The claimed pharmaceutical composition contains non-replicating nanoparticles in the amount of 1.16×1011 virus particle (v.p.) per ml of the formulating buffer. Take the therapeutically effective dosage of non-replicating nanoparticles per 3 ml of the formulating buffer. The dosage form of the preparation might be 1 ml. The preparation dosage form might be 3 ml as well. Isoform 121 of the vascular endothelium growth factor gene is used as the human vascular endothelium growth factor gene.

The method of treatment of amyotrophic lateral sclerosis involves injection to the patient of the therapeutically effective dosage of the pharmaceutical composition containing non-replicating nanoparticles carrying the human angiogenesis factor gene and human vascular endothelium growth factor gene cloned in two expressing cassettes of a single human adenovirus type 5 genome based non-replicating nanoparticle, at the same time the composition additionally contains a formulating buffer. Inject the pharmaceutical composition carrying non-replicating nanoparticles in the amount of 1.16×1011 virus particle (v.p.) per ml of the formulating buffer. The full therapeutically effective dosage of the preparation makes from 3.48×1011 to 7×1013 virus particle (v.p.) per person in a formulating buffer. Perform intramuscular injection of the pharmaceutical composition. Perform injection of the pharmaceutical composition in three muscles (m.trapezius, m.deltoideus, m.quadriceps) bilaterally. Perform injection of the pharmaceutical composition once per 2 weeks for the life term. Conduct start of therapy in 2 stages with increase of the therapeutic dosage, at the first stage inject ⅓ of the full therapeutic preparation dosage in 1 muscle bilaterally. At the second stage inject ⅔ of the full therapeutic preparation dosage, perform injection in 2 muscles bilaterally.

The endogenous angiogenesis factor is known to require induction by other angiogenic proteins, for instance, the vascular endothelial growth factor, for cell proliferation (Kishimoto K. et al., Endogenous angiogenin in endothelial cells is a general requirement for cell proliferation and angiogenesis, Oncogene, 2005, No. 24, p. 445).

The invention is based on the ability of neurotrophic factors—the angiogenesis factor and the vascular endothelium growth factor—to exert a combined positive neurotrophic effect inducing deceleration of motor neurons degeneration.

The human adenovirus type 5 genome was chosen as the vector for production of non-replicating nanoparticles carrying genes of the angiogenesis factor and the vascular endothelium growth factor, which is predetermined by the fact that adenoviral vectors are able to retrograde axonal transport (Boulis N. M. et al., Characterization of adenoviral gene expression in spinal cord after remote vector delivery, Neurosurgery, 1999, No. 45 (1), p. 131-137)

Thus, the unique nature of the claimed pharmaceutical composition as compared to the prototype is presence in the composition of non-replicating nanoparticles on the basis of adenovirus 5 genome representing a vector carrying inserts of two genes: angiogenesis factor and vascular endothelium growth factor in various expressing cassettes.

The stated uniform technical, therapeutic and economic results for implementation of the claimed invention are achieved through production of human neurotrophic proteins—the angiogenesis factor and the vascular endothelial growth factor—in therapeutical concentrations directly in the nervous tissue of the human body by the claimed combined human adenovirus type 5 genome based non-replicating nanoparticle.

The stated uniform technical, therapeutic and economic results on implementation of the claimed invention by the parameter “method of use” are achieved due to the fact that the claimed invention as well as the known method of prevention and/or treatment of neurodegenerative diseases are performed with the help of the recombinant neurotrophic protein—the angiogenesis factor, which was confirmed by preclinical trials. The peculiarity of the claimed method lies in the fact that neurotrophic proteins—the human angiogenesis factor and the human vascular endothelial growth factor—are produced directly in the nervous tissue of the human body following intramuscular injection of the human adenovirus genome based non-replicating nanoparticles, containing two expressing cassettes with the angiogenesis factor and vascular endothelium growth factor gene inserts, and not injected as recombinant proteins or each gene in various vectors, and the start of therapy is carried out at lower dosages as compared to therapeutic dosages. The angiogenesis factor and the vascular endothelial growth factor expressed in the human body exert therapeutic effect as a neurotrophic agent.

The pharmaceutical composition is created for the therapeutic process on the basis non-replicating nanoparticles producing neurotrophic proteins—the angiogenesis factor and the vascular endothelial growth factor.

The pharmaceutical composition is a raw material for production of various dosage forms, application of which is determined depending on the neurotrophic disease. The claimed pharmaceutical composition containing non-replicating nanoparticles with insert of genes encoding human neurotrophic proteins—the angiogenesis factor and the vascular endothelial growth factor—passed clinical trials on investigation of the neurotropic effect, cancirogenic action, general toxic action that proved safety of this composition and the therapeutic activity as a neurotrophic substance illustrated by the following examples.

### BRIEF DESCRIPTION OF THE DRAWINGS

FIG. 1 shows a scheme of two expressing cassettes of the human adenovirus type 5 genome based non-replicating nanoparticle situated in the El deletion area of the adenovirus genome.

FIG. 2 shows PCR results showing expression of the angiogenesis factor gene (shown in light bands) in the spinal cord motor neurons of B6SJL-Tg(SOD1*G93A)dl1 Gur/J mice.

FIG. 3 shows results of PCR showing expression of the vascular endothelium growth factor gene (shown in light bands) in the spinal cord motor neurons of B6SJL-Tg(SOD1*G93A)dl1Gur/J mice.

FIG. 4 shows the data on duration of life of transgenic animals:

### DETAILED DESCRIPTION OF THE PREFERRED EMBODIMENTS

The following tasks for solved for development of the claimed pharmaceutical composition:
design of human adenovirus serotype 5 genome based non-replicating nanoparticles with insert of the angiogenesis factor and vascular endothelium growth factor genes;
development of the method of production of the composition carrying non-replicating nanoparticles that is established on the basis of therapeutic dosages;
determination of the minimum therapeutic dosage and the maximum tolerated dosage.
demonstration of target genes expression in motor neurons;
demonstration of clinical effectiveness and safety of the claimed therapy mode.

The examples represented further disclose the set tasks.

### Example 1

1) Design of a human adenovirus type 5 genome based non-replicating nanoparticle.

2) Production of the pharmaceutical composition.

Expression of the angiogenesis factor and the vascular endothelial growth factor genes required construction of a non-replicating nanoparticle with two-cassette insert. The known recombinant plasmide, for instance pJM17 (Mc Grory W. J., A simple technique for the rescue of early region I mutations into infectious human adenovirus type 5, Virology, No. 163 (2), 1988, c. 614) with deletions in the E1 area of the human adenovirus type 5 genome was used as the basis for construction of the non-replicating particle. Cloning was performed by a homologous recombination in culture cells and was conducted with use of the generally known laboratory methods (for instance, (for example, Sambrook J., Frich E., Maniatis T. et al. Genetic engineering methods. Molecular cloning. M, Mir, 1984, pp. 205-224, 387-420). The artificially synthesized cDNAs of the human angiogenesis factor and the human vascular endothelium growth factor gene were inserted in the known shuttle-vector, for instance, pRcCMV (Invitrogen, San Diego, Calif., No. V75020.) by two successive subclonings by chosen restriction sites. 293 cell culture was co-transfected by the created two-cassette plasmid vector pRcCMV—Ang—VGEF jointly with the pJM17 plasmid (Graham F. L. et al., A new technique for the assay of infectivity of human adenovirus type 5 DNA, Virology, 1973, No. 52 (2), p. 456-467). As a result of the homologous recombination in Line 293 cells (for instance, CLS, Germany, No. 300192,) a non-replicating nanoparticle was produced carrying two expressing cassettes (situated in the E1 deletion area of the human adenovirus genome), each of which contains a cytomegalovirus early area promoter, a target gene (the angiogenesis factor or the vascular endothelium growth factor), a polyadenylation signal, the cassettes are isolated by the vector nucleotide subsequence. After the transfection by the obtained construction of the 293 cell culture the produced plaques were taken by Pasteur pipette and the obtained material was reproduced on Line 293 cells until the titre 3×1010 v.p. (virus particle)/ml (108 PFU per ml) was achieved.

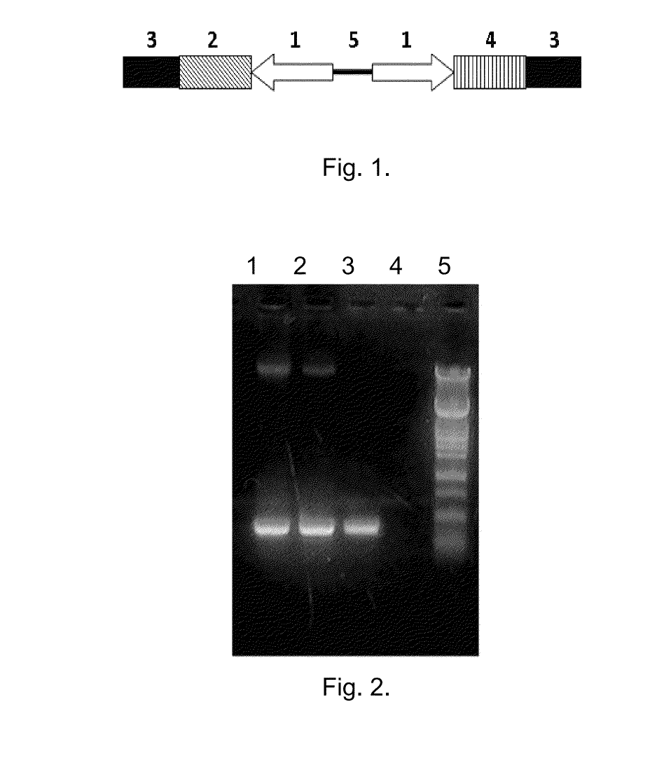

FIG. 1 shows a diagram of two expressing cassettes of a human adenovirus type 5 genome based non-replicating nanoparticle situated in the E1 deletion area of the adenovirus genome. The following keys are used on the diagram:

1—the cytomegalovirus early area promotor ( [symbol] );

2—the angiogenesis factor gene ( [symbol] )

3—the polyadenylation signal ( [symbol] );

4—the vascular endothelium growth factor gene ( [symbol] )

5—the vector nucleotide sequence ( [symbol] )

Thus, a non-replicating nanoparticle on the basis of the human genome virus is designed able to express two neurotrophic human proteins—an angiogenesis factor and a vascular endothelial growth factor.

Production of the Pharmaceutical Composition

On the basis fo the dosages for human use specified in examples 3, 4, 5, 6, the content of non-replicating nanoparticles produced on the basis of human adenovirus type 5 genome in the pharmaceutical composition must be no less than 1.16×1011 pfu/ml (which corresponds to activity no less than 3.3×108 PFU per ml). Production of this composition is conducted in several stages.

The cellular suspension obtained above that carries non-replicating nanoparticles in the titre 3×1010 pfu/ml was used for subsequent increase of titres of non-replicating nanoparticles and for preparation of the pharmaceutical composition with the set content no less than 1.16×1011 pfu/ml (which corresponds to activity no less than 3.3×108 PFU per ml).

A wave bioreactor with 4,500 ml of the permissive cell culture suspension 293 was seeded with a cell suspension with the volume 500 ml carrying non-replicating nanoparticles with titre 3×1010 pfu per ml in order to increase the required non-replicating nanoparticles titre.

Non-replicating nanoparticles were cultivated inside cells until their content achieved 6×1010 pfu per ml (activity 2×108 PFU per ml) approximately for 48 hours in order to increase the titre. On achievement of the required content of nanoparticles the cell mass was fed for purification that included several stages:

1) Settling of the cellular mass by centrifuging. The suspension fed to the centrifuge had no less than 1014 pfu per 51 (evaluated by mass-spectrometer, 1 OU=1012 pfu). Centrifuged the mass in the regime 6,000 g for 15 min, discharged liquid supernatant and fed the remaining solid part containing non-replicating nanoparticles for the subsequent purification stages.

2) Extracted non-replicating nanoparticles from the cell culture means of cell breakage by four-fold freezing-thawing. Prepared a buffer solution with pH 8.0: 5 mMTrisHCI, 0.075 MNaCl, 1 mMMgCl2, 5% saccharose, 1% polisorbat 80. Re-suspended the resulting sediment at the previous stage in 70 ml of the buffer (content ratio ×71). The solution volume amounted to 80 ml.

Froze for 2 hours in the liquid nitrogen, thawed—on the water bath (at +37° C.) preventing overheating.

3) Performed additional nuclease treatment in order to simplify subsequent removal of genome cellular DNA. Added Benzonase until the concentration in the solution made 150 U/ml and set mild mixing with the help of a magnetic stirrer for 3 hours at a room temperature (21-23° C.).

4) Isolated non-replicating nanoparticles from broken cells by centrifuging at 9000 g for 10 min. Collected supernatant carrying non-replicating nanoparticles.

5) Subsequent treatment performed by ultra-filtration. Diluted the resulting supernatant by the buffer buffer OM (50 mM TrisHCl pH 7.5, 1M NaCl, 2 mM MgCl2, 5% saccharose, pH 7,5) until the volume reached no less than 200 ml, mixed by a magnetic stirrer. Permanently made up the volume of the circulating solution (a retentate) to the initial volume (200 ml).

6) Then perfomed purification by anion-exchange chromatography.

Applied the retentate to the column (AxiChrom 70/300 with the volume 400 ml) containing the anion-exchange sorbent Q Sepharose virus licenced. Non-replicating nanoparticles are sorbed on the column whereas admixtures are not sorbed but washed out by buffer A. After removal of admixtures desorbed non-replicating nanoparticles by buffer B washing. Chromatography conditions: flow rate 193 ml/min, buffer A (40 mM TrisHCl, 0.27 M NaCl, 2 mM MgCl2, 5% Saccharose, 0.1% Polysorbate 80, pH 7.5), conductivity ˜28-30 mS/cm; buffer B (40 mM TrisHCl, 0.5M NaCl, 2 mM MgCl2, 5% saccharose, 0.1% polysorbate 80, pH 7.5) conductivity ˜50 mS/cm. Sent eluate in the volume 200 ml ompamisum to the following stage.

7) Exclusive Chromatography

Apply the eluate obtained at the previous stage on the column (AxiChrom 100/300 with the volume 800 ml), containing sorbent Q Sepharose 4 FastFlow. Eluated high-molecular substances not included in the sorbent pores by the first peak (non-replicating nanoparticles belong to them), eluated admixtures after escaping peak of non-replicating nanoparticles. Chromatography conditions: flow rate 130 ml/min, buffer (10 mMTrisHCl, 75 MMNaCl, 1mMMgCl2, 5% saccharose 0.05% Polysorbate 80, pH 8.0).

Added ethanol to the obtained eluate (80 ml) to concentration 0.5% and ethylenediaminetetraacetic acid (EDTA) to concentration 100 μM, sent to the following stage.

8) Normal Filtration

Sterilization of the obtained preparation was performed by filtration through a system of filters with the pore dimension 22 μM. The final volume of the preparation at this stage amounted to 80 ml and contained non-replicating nanoparticles in the titre 1×1012 pfu per ml. Dissolved it by the formulating buffer (for example, 10 mMTrisHCl, 75mMNaCl, 1mMMgCl2, 5% saccharose, 0.05% Polysorbate 80, 0.5% ethanol, 100 μm EDTA, pH 8.0) until the target content 2.33×1011 pfu per ml was obtained and sterilized by normal filtration.

Thus, the above-described method allowed producing the pharmaceutical composition on the basis of the constructed human adenovirus type 5 genome based non-replicating nanoparticles with insert of the angiogenesis factor and the vascular endothelium growth factor genes with the set content no less than 1.16×1011 pfu per ml (which corresponds to activity of the pharmaceutical composition no less than 3.3×108 PFU per ml).

### Example 2

Determination of Angiogenesis Factor and Vascular Endothelium Growth Factor Genes Expression In Vitro

Evaluation of expression by the non-replicating nanoparticles of the angiogenesis factor and the vascular endothelium growth factor was done with the help of the known method ELISA with use of kits (R&D Systems, Quantikine, Human Angiogenin, Cat. No. DAN00; R&D Systems, Quantikine, Human VEGF, Kam No. DVE00) under the producer protocol. The pharmaceutical composition was added to the 293 cell culture so that there was 5-10 Units of non-replicating nanoparticles with inserts of the target genes. The seeding was incubated for 2 days at 37° C. and 5% CO₂, then the culture liquid was taken for analysis. The cultural liquid was used as control obtained after adding to the cell culture of non-replicating nanoparticles without inserts of target genes (Table 1).

TABLE 1

| | Concentration, μg/ml | |
|---|---|---|
| | | vascular endothelial |
| Tested substance | angiogenesis factor | growth factor |
| Cultural medium | 5.8 ± 0.1 | 6.0 ± 0.1 |
| (angiogenesis factor + | | |
| vascular endothelial | | |
| growth factor) | | |
| Cultural medium | Not detected | Not detected |
| (negative control) | | |

Thus, results of Table 1 show presence of the angiogenesis factor and the vascular endothelium growth factor in tested samples of the cultural liquid, which means expression of the angiogenesis factor and the vascular endothelium growth factor by non-replicating nanoparticles with inserts of the angiogenesis factor gene and the vascular endothelium growth factor gene in the form of two cassettes.

Thus, the conclusion was made that the developed pharmaceutical composition produces the angiogenesis factor and the vascular endothelial growth factor in vitro.

### Example 3

### Qualitative Evaluation of Therapeutic Gene Expression in the Spinal Cord

The qualitative evaluation of the therapeutic genes—the angiogenesis factor and the vascular endothelium growth factor—in the motor neurons of the spinal cord after the injection of the pharmaceutical composition at a dosage of 2.15×1011 pfu/sqm to mice of Line B6SJL-Tg(SOD1*G93A)dl1Gur/J (Gurney M. E. et al., Motor neuron degeneration in mice that express a human Cu,Zn superoxide dismutase mutation, Science, 1994, No. 264, p. 1772) (Puschino nursery, Moscow) was performed under the standard PCR (polymerase chain reaction)—amplification of specific cDNA sites from samples of total RNA extracted from lumbar, cervical, thoracic spine of injected mice.

Evaluation of expression of the therapeutic genes—the angiogenesis factor and the vascular endothelium growth factor—was performed on motor neurons of the spinal cord on various stages after the injection of non-replicating nanoparticles carrying the angiogenesis factor and vascular endothelium growth factor gene inserts (Day 4, 8 and 14 after injection).

The RNA kit “Trizol RNA Prep 100” kit (Isogene Laboratory, Russia) was used in accordance with the producer instruction to extract samples of the total RNA from various parts of the spinal cord (lumbar, thoracic, cervical) experimental transgenic mice. Spinal cord samples of 10 mice from experimental group were used for analysis of therapeutic genes—the angiogenesis factor and the vascular endothelium growth factor—in motor neurons of the spinal cord.

Primers for reverse transcription and PCR-amplification of specific cDNA of expressed genes—the angiogenesis factor and the vascular endothelium growth factor—administered through non-replicating nanoparticles: the angiogenesis factor F 5′-gatgacagatactgtgaaagcatcat-3′, angiogenesis factor R 5′-agtggacaggtaagccattttc-3′ (the dimension of the amplification product 280 base pairs) and the vascular endothelial growth factor F 5′-catcacgaagtggtgaagttcat-3′, ΦD [symbol] P3 R 5′-CTTGTCTTGCTCTATCTTTCTTTG-3′ (the dimension of the amplification product-310 base pairs) were chosen and synthesized.

The reverse transcriptase PCR with amplification of specific cDNA sites (angiogenesis factor and vascular endothelium growth factor) was conducted with use of kits “GenePak® RT-PCRCore” (Isogene Laboratory, Russia) in accordance with the producer's protocol. Samples of spinal cord of mice injected by non-replicating nanoparticles without the transgene inserts were used as control samples.

FIG. 2 shows PCR results demonstrating expression of the angiogenesis factor gene (shown in light lines) in spinal cord motor neurons in inkected mice B6SJL-Tg(SOD1*G93A)dl1Gur/J. Lines:

1—an amplification positive line 280 base pairs, a lumbar segment, Day 4 after injection;

2—an amplification positive line 280 base pairs, a lumbar segment, Day 8 after injection;

3—an amplification positive line 280 base pairs, (a positive control);

4—no expression (a negative control);

5—a molecular weight marker (DNA phase λ cut by endonucleases).

Electrophoregrams show the angiogenesis factor gene expression.

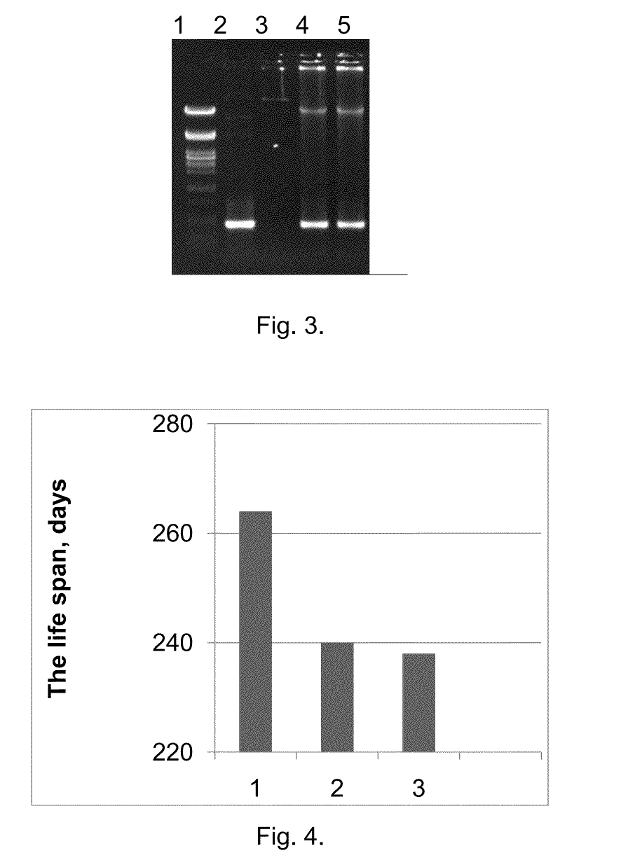

FIG. 3 shows results of PCR demonstrating the vascular endothelium growth factor gene (shown in light lines) in motor neurons of the spinal cord in injected mice B6SJL-Tg(SOD1*G93A)dl1Gur/J

Lines:

1—a molecular weight marker (DNA phage cut by endonucleases).

2—an amplification positive line 310 base pairs, (a positive control);

3—no expression (a negative control);

4—an amplification positive line 310 base pairs, a lumbar segment, Day 4 after injection;

5—amplification positive line 310 base pairs, a lumbar segment, Day 8 after injection;

Electrophoregrams show the vascular endothelium growth factor gene expression.

Analogous results of the expression of the angiogenesis factor and vascular endothelium growth factor genes by non-replicating nanoparticles were obtained in the thoracic and cervical departments of the mice spinal cord.

This study demonstrated expression of angiogenesis factor and vascular endothelium growth factor therapeutic genes in the spinal cord motor neurons on Day 4 after intramuscular injections of the pharmaceutical composition, as well as preservation of the target gene expression on Day 8 after injection. On Day 14 there was no expression of target transgenes (slight lines on the electrophoregram).

Thus, a method of injection of the pharmaceutical composition (repeated intramuscular injections in muscles of fore and hind limbs as well as in spinal muscles) and delivery of therapeutic genes in spinal cord neurons (a retrograde axonal transport) of non-replicating nanoparticles with two expression cassettes with insert of the angiogenesis factor and the vascular endothelium growth factor genes is effective which is proved by the effective expression of the angiogenesis factor and the vascular endothelium growth factor in motor neurons of the spinal cord on Days 4 and 8 after injection of the studied preparations.

Thus, delivery of the acting substance of the pharmaceutical composition following the intramuscular injection at a dosage of 2.15×1011 pfu per sqm in motor neurons of the spinal cord was proved as well as production in them of the angiogenesis factor and the vascular endothelium growth factor.

### Example 4

### Evaluation of Pharmaceutical Composition Clinical Effectiveness on the Basis of Life Duration

A comparative evaluation of the life time of various experimental and control groups of animals showed that the life time of the experimental group of B6SJL-Tg(SOD1*G93A)dl1 Gur/J transgenic mice that received numerous repeated intramuscular injections of the pharmaceutical composition at a dosage of 2.15×1011 pfu/sqm.

FIG. 4 demonstrates the data on life time of transgenic animals:

1—in the experimental mice that received the pharmaceutical composition;

2—a control group that received non-replicating nanoparticles on the basis of the human adenovirus serotype 5 genome (without insert of target genes);

3—a control group that received injections of the buffer.

The figure shows that experimental mice (with symptoms of amyotrophic lateral sclerosis) that received injections of the invented pharmaceutical composition had a considerably higher survival rates (264±20 days) as compared to control groups 2 and 3 (240±14 days and 238±14 days, respectively) that didn't receive constructions expressing the angiogenesis factor and vascular endothelial growth factor.

Thus, injection of the pharmaceutical composition at a dosage of 2.15×1011 pfu/sqm increases the life duration of experimental transgenic animals with the amyotrophic lateral sclerosis model.

### Example 5

In order to calculate the maximum tolerated dosages on mice, female and male animals, a study of toxicity of the pharmaceutical composition following the single intramuscular injection was performed. The dosage 2.15×10¹¹ pfu/sqm was taken as a basis, with the proved effectiveness of retrograde transport in motor neurons of the spinal cord (example 3).

The pharmaceutical composition was injected to animals in the following dosages:
2.15×10¹¹ pfu/sqm, 4.3×10¹¹ pfu/sqm, 43.0×10¹¹ pfu/sqm, 215.0×10¹¹ pfu/sqm, 430.0×10¹¹ pfu/sqm and 860.0×10¹¹ pfu/sqm;

As a result of conducted studies the maximum tolerated and lethal dosage of the human adenovirus serotype 5 for mice following its single intramuscular injection were determined:

For mice:
dosage 430.0×10¹¹ pfu per sqm is characterized as the maximum tolerable dosage;
dosage 860.0×10¹¹ pfu per sqm is characterized as the partially lethal dosage inducing loss of 25% of animals.

No differences in sensitivity of female and male to toxic action of the pharmaceutical composition after its single intramuscular injection were observed.

External signs of intoxication in mice after use of tolerated and maximum tolerated dosage of the preparation were represented by hypodynamia or adynamia, weakness, development of the edema of the limb in the muscle of which the preparation was injected.

Use of the lethal dosage of pharmaceutical composition in mice (860.0×10¹¹ pfu per sqm) resulted in loss of animals on Day 4-18 following injection without clearly marked clinical intoxication signs. Autopsy of lost animals and accurate postmortem examination didn't allow determining the reason of mice death.

Thus, it was concluded by experimental results that the range of tolerated dosages of the pharmaceutical composition in the experiment on determination of the acute toxicity for mice makes from 2.15×10¹¹ pfu per sqm to 430×10¹¹ pfu per sqm.

### Example 6

Determination of the pharmaceutical composition safe administration way.

The developed scheme of administration of the pharmaceutical composition allows preventing local inflammatory post-injection reactions and is based on gradual increase of dosages in the beginning of the therapy. Experimental confirmation of safety of the chosen mode of administration was performed through formation of the experimental and control groups of mice, 10 animals per each.

Experimental mice with B6SJL-Tg(SOD1*G93A)dl1 Gur/J mutation intramuscular injections of the tested pharmaceutical composition, as well as of control solutions was performed in each 2 weeks, starting from the age of 2 months till the end of life. The solution was injected bilaterally in 3 groups of muscles (in fore and hind limbs and the spine), the total volume reached 2.15×10¹¹ pfu/sqm.

The first injection of the pharmaceutical composition to the experimental group of animals was made bilaterally in one of the muscular groups (at option) of fore, hind limbs or spinal muscles, i.e. in 2 points so that ultimately the total dosage of the injected nanoparticles reached 7.17×10¹⁰ pfu/sqm (⅓ of the full dosage). In 2 weeks injection was performed in two muscular groups (at option), i.e. in 4 points so that ultimately the total dosage of injected nanoparticles reached 1.43×10¹¹ pfu/sqm (⅔ of the full dosage). In 2 weeks after injection of partial dosages a therapy by full dosage of the pharmaceutical composition in each of the muscular groups started, i.e. in six points (dosage 2.15×10¹¹ pfu/sqm) once per 2 weeks. Control animals received the preparation in accordance with the regular scheme. The effectiveness of the developed scheme was evaluated clinically—by visual presence of inflammation signs in the injection site, the data are tabulated in Table 2.

TABLE 2

| | Number of animals | Animals with in- |
|---|---|---|
| Group of animals | per group | flammation symptoms |
| pharmaceutical composition | 10 | 0 |
| (experimental) | | |
| pharmaceutical composition | 10 | 9 |
| (control) | | |

The data of Table 2 demonstrate that the preparation injection in accordance with the claimed scheme prevents development of post-injection local inflammations.

The permanent observations of transgenic mice in the course of the experiment on the background of numerous repeated intramuscular injections of the studied pharmaceutical composition signify lack of clinically significant side effects. Repeated intramuscular injections of the pharmaceutical composition don't serve as an intoxication and morbidity factor for all experimental and control groups of transgenic animals.

In all experimental and control groups of transgenic B6SJL-Tg(SOD1*G93A)dl1 Gur/J mice the reaction to injection of the pharmaceutical composition and compared solutions was standard and allowed evaluating the tolerance of the conducted course therapy as favorable:
repeated injections didn't lead to lethal outcomes and rejection of experimental animals;
after injection of the preparation (a comparable solution) animals didn't demonstrate clear signs of allergic reactions or manifestations of systemic intoxication; local inflammatory post-injection reactions were not observed;
no associated pathological states that could be directly or indirectly related to the recombinant preparation injection were observed in experimental groups.

Thus, implementation of the mode of therapy in accordance with the claimed scheme no post-injection local inflammatory reactions after the intramuscular injection of the pharmaceutical composition were observed, which allowed recommending the scheme for human use.

### Example 7

### Method of ALS Therapy

The claimed method of therapy was clinically tested on 6 patients suffering from the cervical and thoracic form of sporadic amyotrophic lateral sclerosis.

Dosages obtained during preclinical studies and measures per sqm of the body surface are equivalent and might be recalculated for human use (the average surface of the human body surface is equal to 1.62 sqm). (Khabriev R. U. Guidance on Experimental (Preclinical) Study of New Pharmacological Substances, 2000, p. 98) (Guidance for Industry. Estimating the Maximum Safe Starting Dose in Initial Clinical Trials for Therapeutics in Adult Healthy Volunteers. U.S. Department of Health and Human Services Food and Medication Administration Center for Medication Evaluation and Research (CDER), July 2005, Pharmacology and Toxicology—pp. 7, 19)

The intramuscular injection of the pharmaceutical composition was performed in 3 muscles (m.trapezius, m.deltoideus, m.quadriceps) from each side each 2 weeks during the life of patient with the purpose of therapy. In order to prevent local inflammatory reactions the dosage of the first injection of the pharmaceutical composition reached 1.16×10¹¹ pfu (⅓ of the full dosage) bilaterally in one of the above-stated muscles, i.e. in 2 points. In 2 weeks intramuscular injection was done in two muscles, i.e. in four points so that finally the total dosage of injected non-replicating nanoparticles amounted to 2.32×10¹¹ pp (⅔ of the full dosage). In 2 weeks more the full dosage of injected preparation made 3.48×10¹¹ pp in each of the muscles bilaterally, i.e. in six points for the life term of the patient.

The effectiveness of the performed scheme of preparation injection aimed at prevention of local post-injection inflammatory reactions was evaluated visually (presence of inflammatory reactions of various degrees in the period between injections). As a result, results on evaluation of 3 post-injection reactions were formed for presence of local inflammatory reactions in patients, the analysis of the obtained data confirmed absence of inflammatory reactions on the first, second and third preparation injection. In the delayed period of treatment no inflammatory response to a successive injection of the full antigen dosage was registered (during a year).

Thus, a scheme of the pharmaceutical composition injection based on gradual increase of dosages at the beginning of therapy allows conducting treatment of amyotrophic lateral sclerosis without any side effects in the site of the preparation injection.

Advantages of the pharmaceutical composition produced under the invention include:
presence in one non-replicating nanoparticle of two inserts—the angiogenesis factor and the vascular endothelium growth factor gene;
reduction of production costs, content of non-replicating nanoparticles in a single dosage is reduced by two-fold,
reduction of the preparation reactigenicity due to reduction of non-replicating nanoparticles quantity;
an injection once per two weeks in order to obtain the therapeutic concentration of target proteins for the human body;
prolonged action (for 2 weeks);
reduction of medication and medical instruments consumption, working time of medical personnel;
reduction of production costs;
symptoms relief;
extension of the patient life time;
a safe range of dosages for human use is known.

### INDUSTRIAL APPLICABILITY

Use in the pharmaceutical and clinical practice of the claimed pharmaceutical composition and methods of its application allows achieving several technical, therapeutic and economic results:
the claimed pharmaceutical composition is biocompatible with the human body and is effective from the therapeutical point of view;
the claimed pharmaceutical composition is economically advantageous as a combined construction of non-replicating nanoparticles containing two genes—the angiogenesis factor and the vascular endothelium growth factor—is used, which allows reduction of the dosage of the non-replicating nanoparticles by two-fold for achievement of the therapeutic effect as compared to compositions containing inserts of these genes in vectors separately;
the claimed pharmaceutical composition is biologically safe for the human body—it doesn't induce aggravation of the disease, it is not cancirogenic neither toxic, it doesn't cause local and general post-injection reactions;
the pharmaceutical composition is suitable for intramuscular injection;
the pharmaceutical composition is suitable for therapy of neurodegenerative diseases, ALS, in particular;
the pharmaceutical composition is convenient for application as it's injected once per 2 weeks;
on a long-term basis (i.e. for 2 weeks) produces in the human body neurotrophic proteins—the angiogenesis factor and the vascular endothelial growth factor—creating in the body the concentration that by ten-fold exceeds the normal level and the concentration required for achievement of stable therapeutic effect;
application of the pharmaceutical composition is economically justified as injection once per 2 weeks provides therapeutic effect during all this time.
the preparation is suitable for production of medications intended for therapy of neurotropic localization diseases;
the preparation is convenient in storage and transportation as well as in application.

The pharmaceutical composition by invention might be represented by drugs represented by a solution of non-replicating nanoparticles for intramuscular injection.

As a result of the invention implementation of the claimed method of therapy with the aim of the claimed pharmaceutical composition allows during intramuscular injection expressing target neuroprotective proteins—the angiogenesis factor and the vascular endothelial growth factor—in therapeutically effective amounts directly in damaged motor neurons of the spinal cord in patients affected by neurodegenerative disease (amyotrophic lateral sclerosis) and increasing their life time. The dosage form 1 and 3 ml allows injecting the composition with increase of the dosage at the beginning of therapy for prevention of any post-injection inflammatory reasons, which allows safely continuing the therapy during the whole life time of the patient. This testifies the industrial applicability and achievement of tasks set in this invention.

What is claimed is:

1. A pharmaceutical composition for therapy of neurodegenerative diseases including amyotrophic lateral sclerosis, the composition comprising:
an adenoviral vector, expressing a human angiogenesis factor gene;
an effective amount of the adenoviral vector in the form of a non-replicating nanoparticle based on a genome of human adenovirus type 5 with an insertion of a human angiogenin gene which produces the human angiogenesis in a human body;
a non-replicating nanoparticle based on the genome of human adenovirus type 5 with an insertion of a human vascular endothelium growth factor gene; and
a formulating buffer;
wherein the human angiogenin factor gene and the human vascular endothelium growth factor gene are cloned in two expression cassettes within one non-replicating nanoparticle based on the genome of human adenovirus type 5.

2. The pharmaceutical composition of claim 1, wherein the formulating buffer comprises about 1.16×10¹¹ virus particle (v.p.) of the non-replicating nanoparticles per ml of the buffer.

3. The pharmaceutical composition of claim 2, wherein a therapeutically effective dosage of the non-replicating nanoparticles is taken per 3 ml of the formulating buffer.

4. The pharmaceutical composition of claim 2, wherein the pharmaceutical form is the form of a 1 ml dose.

5. The pharmaceutical composition of claim 2, wherein the pharmaceutical form is in the form of a 3 ml dose.

6. The pharmaceutical composition of claim 1, wherein the human vascular endothelium growth factor gene is a vascular endothelial growth factor of isoform 121.

7. A method of therapy of amyotrophic lateral sclerosis comprising injecting a therapeutically effective dosage of a pharmaceutical composition comprising a formulating buffer and non-replicating nanoparticles comprising a human angiogenin factor gene and a human vascular endothelium growth factor gene cloned in two expression cassettes with one non-replicating nanoparticle based on a genome of human adenovirus type 5.

8. The method of claim 7, wherein the formulating buffer comprises about 1.16×10¹¹ virus particle (v.p.) of the non-replicating nanoparticles per ml of the buffer.

9. The method of claim 8, wherein a full therapeutically effective dosage of the pharmaceutical composition per human ranges from 3.48×10¹¹ to 7×10¹³ virus particle (v.p.) per in the formulating buffer.

10. The method of claim 7, wherein injecting is done intramuscularly.

11. The method of claim 10, wherein injecting is done into three muscles.

12. The method of claim 11, wherein injecting is done bilaterally into m.trapezius, m.deltoideus and m.quadriceps.

13. The method of claim 7, wherein the pharmaceutical composition is injected once per two weeks.

14. The method of claim 13, wherein the pharmaceutical composition is injected over a lifetime of a patient.

15. The method of claim 7, wherein the start of the therapy is performed in two stages with an increase of the therapeutically effective dosage.

16. The method of claim 15, wherein ⅓ of the therapeutically effective dosage is injected at a first stage.

17. The method of claim 16, wherein injecting is done bilaterally into one muscle.

18. The method of claim 15, wherein ⅔ of the therapeutically effective dosage is injected at a second stage.

19. The method of claim 18, wherein injecting is done bilaterally into two muscles.
